(19) United States
(12) Patent Application Publication
(10) Pub. No.: US 2013/0274216 A1
(43) Pub. Date: Oct. 17, 2013

(54) TREATMENT FOR MICROBE-INDUCED INFLAMMATORY RESPONSES IN THE EYE

(71) Applicant: ALCON RESEARCH, LTD., Fort Worth, TX (US)

(72) Inventors: Eric C. Carlson, Burleson, TX (US); Mark R. Hellberg, Arlington, TX (US); John M. Yanni, Burleson, TX (US)

(73) Assignee: ALCON RESEARCH, LTD., Fort Worth, TX (US)

(21) Appl. No.: 13/861,603

(22) Filed: Apr. 12, 2013

Related U.S. Application Data
(60) Provisional application No. 61/623,320, filed on Apr. 12, 2012.

(51) Int. Cl. A61K 31/519; A61K 31/7048

(52) U.S. Cl. CPC A61K 31/519; A61K 31/7048
USPC 514/31

(57) ABSTRACT

The present invention relates to ophthalmic compositions comprising inhibitors of spleen tyrosine kinase (syk). The compositions are particularly well suited for the treatment of ophthalmic infection such as fungal keratitis. The compositions optionally comprise an antiinfective compound such as an antibacterial or antifungal compound. The present invention also relates to methods for treating fungal keratitis using compositions comprising syk inhibitors.

### CROSS-REFERENCE TO RELATED APPLICATION

This application claims priority under 35 U.S.C. §119 to U.S. Provisional Patent Application No. 61/623,320 filed Apr. 12, 2012 the entire contents of which are incorporated herein by reference.

### TECHNICAL FIELD OF THE INVENTION

The present invention relates generally to compounds useful for the treatment of ophthalmic inflammation induced by infections, particularly inflammation as a result of fungal infection. The present invention particularly relates to compositions comprising inhibitors of spleen tyrosine kinase (syk) and methods of using such compositions for the treatment of infection-induced ophthalmic inflammation.

### BACKGROUND OF THE INVENTION

Persistent corneal inflammation secondary to microbial infection is a serious threat to visual acuity due to the breakdown of corneal epithelial integrity and the cortical stromal arrangement. This breakdown occurs due to the acute and significant infiltration of inflammatory cells (primarily neutrophils) in response to cytokine expression by resident corneal cells (corneal epithelium, keratocytes, resident macrophages and dendritic cells). In the context of fungal infection, the expression of cytokines is initiated following recognition of the infection by resident corneal cells via pathogen recognition molecules, which include C-type lectins (e.g., dectin-1 and dectin-2) that recognize β-glucan, a component of the fungal cell wall. This signaling pathway leads to the recruitment and activation of inflammatory cells that initiate and elevate the innate inflammatory response.

Ocular inflammation has historically been treated with a regimen of oral, intranasal or topical antihistamines, or oral or intranasal steroids, or, in the case of allergy, allergen injection treatment. Systemic treatment typically requires higher concentrations of the drug compound to be administered to afford an effective concentration to reach the necessary treatment site. Antihistamine compounds are known to have central nervous system activity; however, drowsiness and drying of mucus membranes are a common side-effect of antihistamine use. Steroid compounds are known to adversely impact intraocular pressure when applied topically, and can cause lens opacification side-effects and a reduction in epithelial cell migration that can affect, for example, wound closure. Accordingly, immune suppression induced by steroids may have adverse consequences during an infection.

Infections of ocular tissue by fungi such as Aspergillus flavus and Fusarium solani cause fungal keratitis, and such infections affect many thousands of people yearly. Left untreated, this disorder causes corneal damage and blindness in almost all affected patients due to the resultant inflammation. Even with current treatments, up to 35 percent of fungal keratitis cases will require corneal grafting or transplant and over 50 percent of patients will be left with impaired vision due to the robust influx of inflammatory cells. Tanure, et al., “Spectrum of Fungal Keratitis at Wills Eye Hospital, Philadelphia, Pa.” Cornea 19(3):307-312 (2000). Current drug treatments have substantial side-effects and further compromise the integrity and wound healing response of the cornea, which increases the risk for secondary infection and fibrosis. Steroid treatment of the inflammatory response is counter indicated until the infectious organism has been effectively killed in ocular tissue. New treatments for fungal keratitis are accordingly desirable.

### BRIEF SUMMARY OF THE INVENTION

The invention relates to inhibitors of spleen tyrosine kinase (syk), and the use of such inhibitors to treat ophthalmic inflammation induced by microbial infections, particularly fungal infections such as fungal keratitis. The present inventors have unexpectedly discovered that the syk inhibitors of the present invention can reduce inflammation associated with microbial infections such as fungal keratitis, while minimizing suppression of natural immune defenses against such infection. Prevention or resolution of the inflammatory response and inflammatory cell infiltration can preserve corneal clarity and visual function in the context of microbial infection of ocular tissues.

In one embodiment, the invention provides a method for treating inflammation associated with ocular infections such as fungal keratitis by administering a composition comprising a syk kinase inhibitor. Optionally, such compositions can comprise an antiinfective such as an antifungal compound. Other ocular inflammation-associated infections, including but not limited to conjunctivitis, keratitis, endophthalmitis, and blepharitis can also be treated using the compositions of the present invention.

In another embodiment, the invention provides compositions for the treatment of fungal keratitis comprising a syk inhibitor and an antifungal compound such as natamycin.

The response to microbial infection of ocular tissues such as the cornea involves a robust and acute infiltration of neutrophils and macrophages. Infiltration and degranulation of neutrophils can result in significant corneal scarring, ulceration and, in the case of fungal infection, facilitates further infiltration of fungal hyphae into the anterior segment of the eye. In yet another embodiment, the invention provides a method of reducing neutrophil and macrophage infiltration of infected ocular tissue by treating said tissue with a composition comprising a syk kinase inhibitor and, optionally, an antiinfective such as an antifungal compound. In preferred embodiments, the compositions are suitable for topical application.

### BRIEF DESCRIPTION OF THE DRAWINGS

A more complete understanding of the present invention and the advantages thereof may be acquired by referring to the following description, taken in conjunction with the figures of the accompanying drawing in which like reference numbers indicate like features and wherein:

FIG. 1 shows micrographs showing the effects of fungal infection of ocular tissue in a murine model of fungal keratitis;

FIG. 2 is a chart comparing neutrophil infiltration of Fusarium condida infected murine corneal tissue in groups treated with a syk inhibitor composition compared to control and vehicle groups;

FIG. 3A shows fluorescence micrographs of control and treatment murine corneas taken 72 hours after infection with Aspergillus flavus; and

FIG. 3B shows a chart presentation comparing CFU/eye of Aspergillus flavus recovered from murine corneas taken 72 hours after infection.

### DETAILED DESCRIPTION OF THE INVENTION

Compositions of the present invention comprise a compound having an inhibitory effect on the expression or activity of spleen tyrosine kinase (syk). These syk inhibitors include, but are not limited to the following compounds:

2-[7-(3,4-dimethoxyphenyl)-imidazo[1,2-c]pyrimidin-5-ylamino]-nicotinamide dihydrochloride, 2-(2-aminoethylamino)-4-(3-methylanilino) pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide, NVP-QAB205, piceatannol, 4-(3-(2H-1,2,3-triazol-2-yl)phenylamino)-2-((1R,2S)-2-aminocyclohexylamino)pyrimidine-5-carboxamide acetate (P505-15), and 3,4-dimethyl-10-(3-aminopropyl)-9-acridone oxalate.

Two particularly preferred syk inhibitors are:

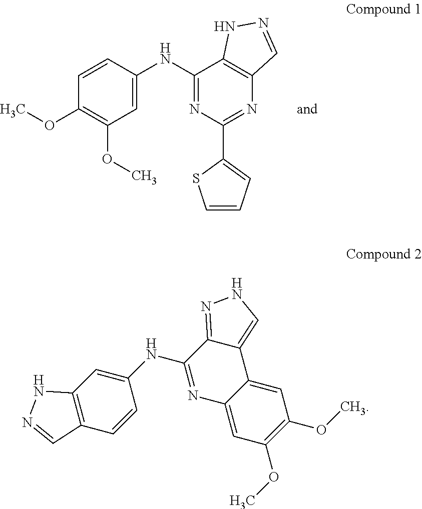 [SMILES: COc1ccc(Nc2nc(-c3cccs3)nc3cn[nH]c23)cc1OC ; COc1cc2nc(Nc3ccc4cn[nH]c4c3)c3n[nH]cc3c2cc1OC]

Additional exemplary syk inhibitors of the present invention are found in the following publications, all of which are herein incorporated by reference in their entirety: U.S. Pat. No. 7,671,063 entitled “2,4 Di (hetero)-arylamino-pyrimidine derivatives as ZAP-70 and/or syk inhibitors”; U.S. Patent Publication No. 2008/0139531 entitled “Use of connective tissue mast cell stabilizers to facilitate ocular surface re-epithelization and wound repair”; U.S. Pat. No. 8,063,058 entitled “Inhibitors of syk and JAK protein kinases”; U.S. Pat. No. 8,138,339 entitled “Inhibitors of protein kinases”; U.S. Patent Publication No. 2011/0053897 entitled “Compounds and compositions as syk kinase inhibitors”; WO2011/014795 entitled “Compounds and compositions as syk kinase inhibitors”; WO2010/146133 entitled “Heterocyclylaminopyrimidines as kinase inhibitors”; WO2010/097248 entitled “Pyrimidinecarboxamide derivatives as inhibitors of syk kinase”; U.S. Patent Publication No. 2010/0222323 entitled “Imidazopyrazine syk inhibitors”; U.S. Patent Publication No. 2010/0093698 entitled “Aminotriazolopyridines, compositions thereof, and methods of treatment therewith”; U.S. Patent Publication No. 2011/0201608 entitled “Substituted naphthyridines and use thereof as medicines”; U.S. Patent Publication No. 2011/0112098 entitled “Molecules inhibiting a metabolic pathway involving the syk protein tyrosine kinase and method for identifying said molecules”; U.S. Patent Publication No. 2012/0329780 entitled “Novel kinase inhibitors” and U.S. Patent Publication No. 2012/0329785 entitled “Novel kinase inhibitors”.

Additional syk inhibitors are disclosed in Drug Discovery Today Vol. 201:15(13/14):517-530. Furthermore, syk inhibitors can be identified using in vitro or in vivo assays known to those of skill in the art.

It is contemplated that the concentration of the syk inhibitor in the compositions of the present invention can vary, but is preferably 0.01 to 1.0 w/v % and more preferably 0.05-0.5 w/v %. The most preferred concentration range is from 0.05-0.3 w/v % and the most preferred concentration is about 0.1 w/v %. The syk inhibitors of the present invention comprise the pharmaceutically useful stereoisomers of above described compounds, as well as the pharmaceutically useful hydrates and salts of such compounds and stereoisomers, and may be formulated with a pharmaceutically acceptable vehicle.

The invention is particularly directed toward treating mammalian and human subjects having or at risk of having a microbial tissue infection. Embodiments of the present invention are particularly useful for treating ophthalmic tissue inflammation and infections. Inflammation as a result of infections of the eye can occur in all ocular tissues or fluids, and include diseases of the lid or lid margins (e.g., blepharitis), the conjunctiva (conjunctivitis), the cornea (e.g., microbial keratitis) and the deeper intraocular fluids or tissues (e.g., endophthalmitis).

Compositions of the present invention may be utilized in various dosage regimens known to those of skill in the art. Such dosing frequency is maintained for a varying duration of time depending on the therapeutic regimen. The duration of a particular therapeutic regimen may vary from one-time dosing to a regimen that extends for a month or more. One of ordinary skill in the art would be familiar with determining a therapeutic regimen for a specific indication. Factors involved in this determination include the disease to be treated, particular characteristics of the subject, and the particular antimicrobial composition. Preferred dosage regimens of the present invention include, but are not limited to, once a day dosing, twice a day dosing, and three times a day dosing.

In the methods set forth herein, administration to a subject of a composition of the present invention may be by various methods known to those of skill in the art, including, but not limited to, topical, subconjunctival, periocular, retrobulbar, subtenon, intraocular, subretinal, posterior juxtascleral, or suprachoroidal administration. In preferred embodiments, administration of a composition of the present invention is by topical administration to the ocular surface.

The compositions of the present invention may optionally comprise in addition to a syk inhibitor an anti-infective compound such as an antibacterial, antifungal, or antiviral compound. Such compounds include, but are not limited to, fluoroquinolones such as moxifloxacin, finatioxacin, ciprofloxacin and other anti-infective compounds known to those of skill in the art. Anti-fungal compounds such as natamycin, voriconazole, tolnaftate, butenafine, ciclopirox, clotrimazole, enconazole, ketoconazole, miconazole, naftifine, nystatin, oxiconazole, terbinafine, amphotericin B, flucytosine, ketoconazole, fluconazole, and itraconazole are particularly preferred.

In addition to a syk inhibitor, the compositions of the present invention optionally comprise one or more excipients. Excipients commonly used in pharmaceutical compositions include, but are not limited to, tonicity agents, preservatives, chelating agents, buffering agents, surfactants and antioxidants. Other excipients comprise solubilizing agents, stabilizing agents, comfort-enhancing agents, polymers, emollients, pH-adjusting agents and/or lubricants. Any of a variety of excipients may be used in compositions of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the syk inhibitor. In preferred embodiments, excipients are selected on the basis of their inertness towards the syk inhibitor.

Relative to ophthalmic formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68. Suitable antioxidants include, but are not limited to, sulfites, ascorbates, butylated hydroxyanisole (BHA) and butylated hydroxytoluene (BHT).

The compositions set forth herein may comprise one or more preservatives. Examples of such preservatives include p-hydroxybenzoic acid ester, sodium chlorite, benzalkonium chloride, parabens such as methylparaben or propylparaben, alcohols such as chlorobutanol, benzyl alcohol or phenyl ethanol, guanidine derivatives such as polyhexamethylene biguanide, sodium perborate, or sorbic acid. In certain embodiments, the composition may be self-preserved that no preservation agent is required.

In preferred compositions a syk inhibitor of the present invention will be formulated for topical application to the eye in aqueous solution in the form of drops. The term “aqueous” typically denotes an aqueous composition wherein the composition is >50%, more preferably >75% and in particular >90% by weight water. These drops may be delivered from a single dose ampoule which may preferably be sterile and thus render bacteriostatic components of the composition unnecessary. Alternatively, the drops may be delivered from a multi-dose bottle which may preferably comprise a device which extracts any preservative from the composition as it is delivered, such devices being known in the art.

In other aspects, components of the invention may be delivered to the eye as a concentrated gel or a similar vehicle, or as dissolvable inserts that are placed beneath the eyelids. In yet other aspects, components of the invention may be delivered to the eye as ointments, water-in-oil and oil-in-water emulsions, solutions, or suspensions.

The compositions of the present invention, and particularly the topical compositions, are preferably isotonic or slightly hypotonic in order to combat any hypertonicity of tears caused by evaporation and/or disease. This may require a tonicity agent to bring the osmolality of the composition to a level at or near 210-320 milliosmoles per kilogram (mOsm/kg). The compositions of the present invention generally have an osmolality in the range of 220-320 mOsm/kg, and preferably have an osmolality in the range of 235-300 mOsm/kg. The ophthalmic compositions will generally be formulated as sterile aqueous solutions.

In certain embodiments, a syk inhibitor of the present invention is formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; guars, such as HP-guar and other guar derivatives, and carbomers, such as carbomer 934P, carbomer 941, carbomer 940 and carbomer 974P. Certain compositions of the present invention may be used with contact lenses or other ophthalmic products.

In certain embodiments, the compositions set forth herein have a viscosity of 0.5-100 cps, preferably 0.5-50 cps, and most preferably 1-20 cps. These viscosities insure that the product is comfortable, does not cause blurring, and is easily processed during manufacturing, transfer and filling operations.

The syk inhibitors described herein may be included in various types of compositions having activities in addition to antiinflammatory activity. Examples of such compositions include: ophthalmic pharmaceutical compositions, such as ocular lubricating products, artificial tears, astringents, topical disinfectants (alone or in combination with other antimicrobial compounds such as, for example, betadine, etc.) and so on.

Preferred compositions are prepared using a buffering system that maintains the composition at a pH of about 3 to a pH of about 8.0, preferably 5.5-7.5, and most preferably 6.0-7.4. Topical compositions (particularly topical ophthalmic compositions) are preferred which have a physiological pH matching the tissue to which the composition will be applied or dispensed.

The following examples are presented to further illustrate selected embodiments of the present invention.

### Example 1

| | Concentration |
|---|---|
| Ingredient | (w/v %) |
| Syk Inhibitor | 0.1% |
| Dibasic Sodium Phosphate | 0.2% |
| Sodium Chloride | 0.75% |
| Disodium EDTA | 0.01% |
| Polysorbate 80 | 0.05% |
| Benzalkonium Chloride Solution | 0.01% |
| Hydroxypropyl Methylcellulose | 0.5% |

### Example 2—In Inhibition of Syk

Compounds can be tested for the ability to inhibit syk-catalyzed phosphorylation of a peptide substrate in a biochemical fluorescence polarization assay with isolated syk kinase. Test compounds are diluted to 1% DMSO in kinase buffer (20 mM HEPES, pH 7.4, 5 mM MgCl₂, 2 mM MnCl.sub.2, 1 mM DTT, 0.1 mg/mL acetylated Bovine Gamma Globulin). Compounds in 1% DMSO (0.2% DMSO final) are mixed with ATP/substrate solution at room temperature. Syk kinase (Upstate, Lake Placid N.Y.) is added to a final reaction volume of 20 uL, and the reaction is incubated for 30 minutes at room temperature Final enzyme reaction conditions are 20 mM HEPES, pH 7.4, 5 mM MgCl.sub.2, 2 mM MnCl.sub.2, 1 mM DTT, 0.1 mg/mL acetylated Bovine Gamma Globulin, 0.125 ng Syk, 4 uM ATP, 2.5 uM peptide substrate (biotin-EQEDEPEGDYEEVLE-CONH2, SynPep Corporation). EDTA (10 mM final)/anti-phosphotyrosine antibody (1× final)/fluorescent phosphopeptide tracer (0.5× final) is added in FP Dilution Buffer to stop the reaction for a total volume of 40 uL according to manufacturer's instructions (PanVera Corporation). The plate is incubated for 30 minutes in the dark at room temperature. Plates are read on a Polarion fluorescence polarization plate reader (Tecan). Data was converted to an amount of phosphopeptide present using a calibration curve generated by competition with the phosphopeptide competitor provided in the Tyrosine Kinase Assay Kit, Green (PanVera Corporation).

### Example 3—In Vivo Fungal Keratitis Model

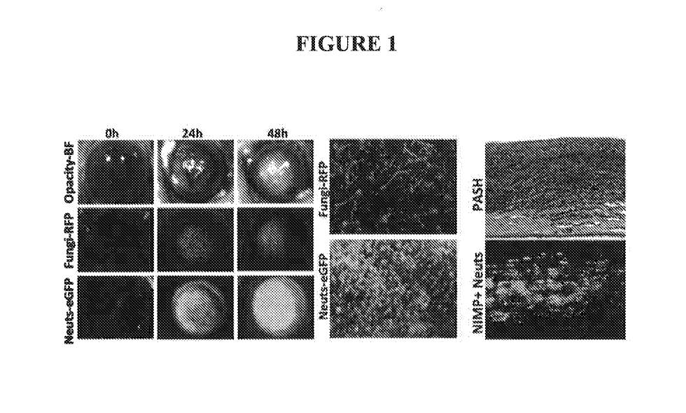

A murine model of fungal keratits was used to test the syk inhibitor compositions of the present invention. The model is similar to the model described by Tarabishy et al. (J Immunol; Vol. 181:593-600; 2008, incorporated herein by reference in its entirety). Briefly, C57BL/6 mice are inoculated with Fusarium conidia via intrastromal injection following corneal abrasion. Mice are examined under a stereomicroscope for ocular opacity. FIG. 1, left top panel shows mouse eyes under normal light showing progression of opacity as fungal infection spreads over 48 h. Fusarium was modified to express red fluorescent protein (RFP) using standard techniques. (FEMS Microbiol Letters; Vol. 225(2):305-9; 2003 Aug. 29, incorporated by reference in its entirety). FIG. 1, left middle panel shows that the RFP expression corresponds with the opacity in the top panel. Neutrophil expression was examined using anti-mouse neutrophil (NIMP) antibody conjugated with green fluorescent protein (GFP). FIG. 1, left bottom panel shows that neutrophil expression increased dramatically as the fungal infection spread throughout the cornea. Histology sections shown in the middle and right panels of FIG. 1 also confirm that fungi and neutrophils were found throughout the thickness of the infected murine cornea.

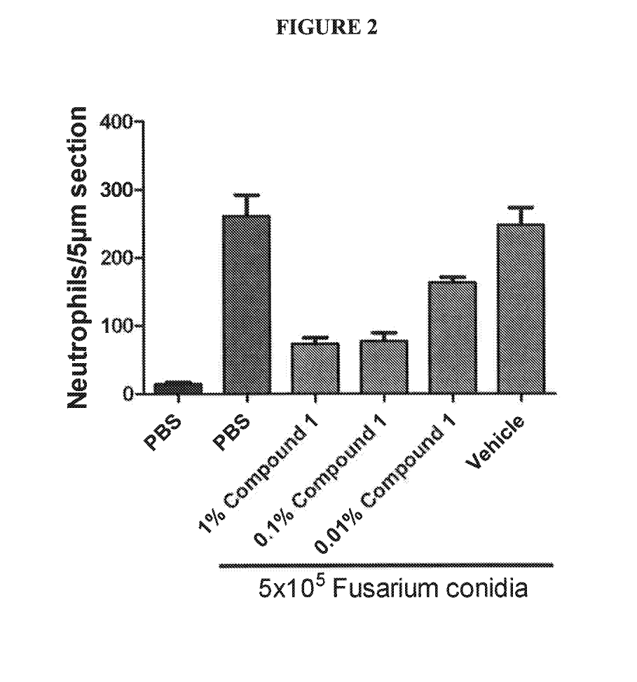

FIG. 2 presents data comparing the neutrophil infiltration in control and test groups of animals. Neutrophil infiltration is assessed after 24 hours by counting neutrophils per 5 μm corneal section. Virtually no neutrophils were found in an uninfected group treated with topical PBS alone. Infected groups treated with PBS or vehicle both had substantial neutrophil infiltration (>200 neutrophils/section). A dose-dependent reduction in neutrophil infiltration was observed in test groups treated with the syk inhibitor Compound 1, with relatively minor elevations in neutrophil count (<100 neutrophils/section) observed relative to control in the 1% and 0.1% w/v test groups.

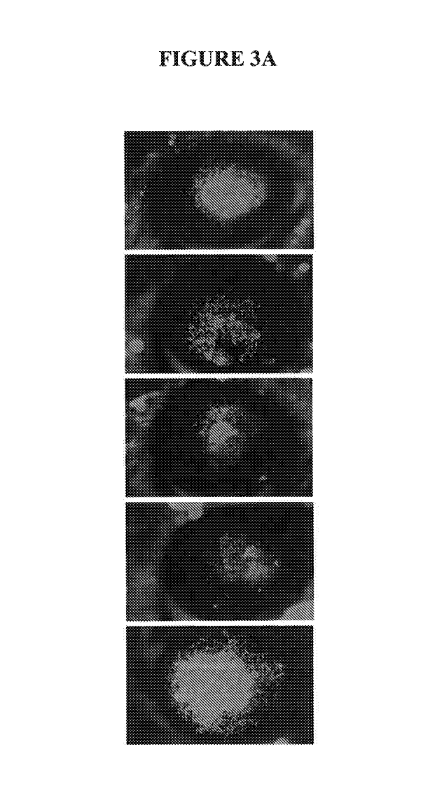

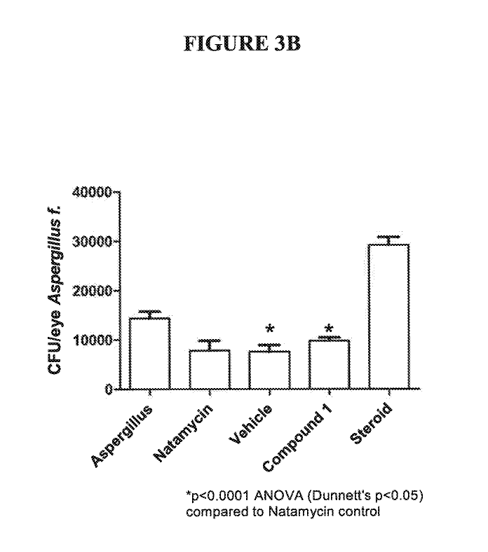

FIGS. 3A & 3B shows data showing that a syk inhibitor of the present invention (Compound 1) can be co-administered with the antifungal compound natamycin. As shown in FIG. 3B, 72 hours after infection with Aspergillus, the control group had approximately 15000 CFUs/eye. In the natamycin treatment group (dosed qid for 48 hours) a nearly 50% reduction in CFUs/eye was observed. A similar reduction was found for a group that was treated with natamycin and vehicle and for a group treated with natamycin and Compound 1. Natamycin co-administration with the anti-inflammatory steroid dexamethasone was associated with a large increase in CFUs per eye. FIG. 3A shows that Aspergillus infection (evaluated by RFP expression) was suppressed in the natamycin, natamycin/vehicle, and natamycin/Compound 1 groups, while the natamycin/dexamethasone group showed increased evidence of infection.

All publications and patent applications cited in this specification are herein incorporated by reference as if each individual publication or patent application were specifically and individually indicated to be incorporated by reference. The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A pharmaceutical composition comprising a syk inhibitor and an antimicrobial compound.

2. A pharmaceutical composition according to claim 1 wherein said syk inhibitor is selected from the group consisting of:
2-[7-(3,4-dimethoxyphenyl)-imidazo[1,2-c]pyrimidin-5-ylamino]-nicotinamide dihydrochloride, 2-(2-aminoethylamino)-4-(3-methylanilino) pyrimidine-5-carboxamide, 2-(2-aminoethylamino)-4-(3-trifluoromethylanilino)primidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrmidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)primidine-5-carboxamide, NVP-QAB205, piceatannol, 4-(3-(2H-1,2,3-triazol-2-yl)phenylamino)-24(1R,2S)-2-aminocyclohexylamino)pyrimidine-5-carboxamide acetate (P505-15), and 3,4-dimethyl-10-(3-aminopropyl)-9-acridone oxalate.

3. A pharmaceutical composition according to claim 1 wherein said syk inhibitor is selected from the group consisting of:

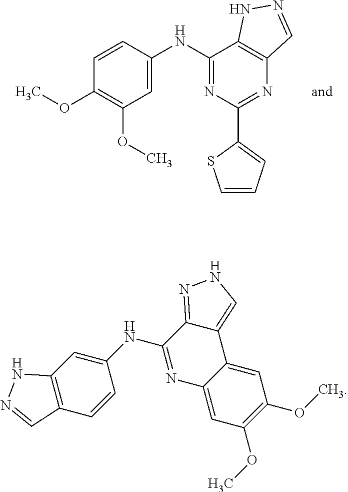 [SMILES: COc1ccc(Nc2nc(-c3cccs3)nc3cn[nH]c23)cc1OC ; COc1cc2nc(Nc3ccc4cn[nH]c4c3)c3n[nH]cc3c2cc1OC]

4. A pharmaceutical composition according to claim 3 wherein said antimicrobial is natamycin.

5. A pharmaceutical composition according to claim 1, wherein said syk inhibitor is present at a concentration of from 0.05% to 0.3% w/v.

6. A pharmaceutical composition according to claim 1, wherein said syk inhibitor is present at a concentration of about 0.1% w/v.

7. A method of treating ophthalmic infections, which comprises administering a pharmaceutically effective amount of the composition of claim 1 to an ophthalmic tissue.

8. A method of treating ophthalmic infections, which comprises topically applying a pharmaceutically effective amount of the composition of claim 4 to the affected ophthalmic tissue.

9. A method according to claim 7, wherein said antimicrobial compound is selected from the group consisting of:
anti-fungal compounds.

10. A method according to claim 7, wherein said antimicrobial compound is selected from the group consisting of:
natamycin, voriconazole, tolnaftate, butenafine, ciclopirox, clotrimazole, enconazole, ketoconazole, miconazole, naftifine, nystatin, oxiconazole, terbinafine, amphotericin B, flucytosine, ketoconazole, fluconazole, and itraconazole.

11. A method according to claim 7, wherein said anti-microbial compound is natamycin.

12. A method according to claim 7, wherein the ophthalmic infection is a fungal infection.

13. A method according to claim 7, wherein the ophthalmic infection is fungal keratitis or fungal endophthalmitis.

14. A method of reducing neutrophil infiltration of infected ocular tissue, said method comprising:
treating said infected tissue with a composition comprising a syk kinase inhibitor.

15. A method according to claim 14, wherein said composition additionally comprises an antiinfective compound.

16. A method according to claim 14, wherein said syk kinase inhibitor is selected from the group consisting of:

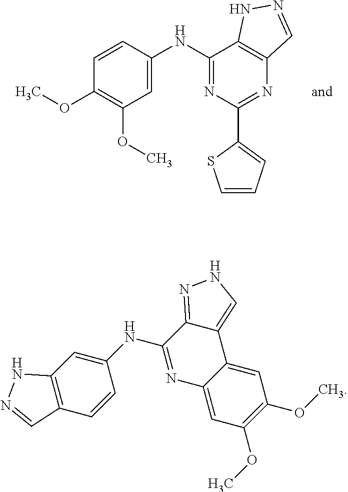 [SMILES: COc1ccc(Nc2nc(-c3cccs3)nc3cn[nH]c23)cc1OC ; COc1cc2nc(Nc3ccc4cn[nH]c4c3)c3n[nH]cc3c2cc1OC]

17. A method according to claim 16, wherein said antiinfective compound is natamycin.

18. A method according to claim 14, wherein said composition is a topical composition.

19. A method according to claim 14, wherein the ocular tissue infection is fungal keratitis or fungal endophthalmitis.
